(19) United States
(12) Patent Application Publication
(10) Pub. No.: US 2006/0020040 A1
(43) Pub. Date: Jan. 26, 2006

(54) Bupropion hydrochloride solid dosage forms

(76) Inventors: Manish Chawla, Rohini (IN); Rajeev Singh Raghuvanshi, New Delhi (IN); Ashok Rampal, Amritsar (IN)

Correspondence Address: Jayadeep R. Deshmukh;Ranbaxy Pharmaceyticals, Inc., 600 College Road East, Suite 2100, Princeton, NJ, 08540, US

(21) Appl. No.: 10/534,910

(22) PCT Filed: Nov. 17, 2003
(86) PCT No.: PCT/IB03/05195

§ 371 Date: May 13, 2005

(30) Foreign Application Priority Data
Nov. 15, 2002 (IN) 1156/DEL/2002

(51) Int. Cl. A61K 31/138

(52) U.S. Cl. 514/649

(57) ABSTRACT

The present invention relates to solid dosage forms that contain bupropion hydrochloride and glucono delta lactone or its corresponding open chain hydroxy acid derivative. The bupropion hydrochloride retains at least 80% of the bupropion hydrochloride potency after storage for three months at 40° C. and 75% relative humidity. The solid dosage form may be in the form of a tablet, a capsule, or a granulate with or without an immediate release profile, a modified release profile, or an extended release profile.

### FIELD OF THE INVENTION

The present invention relates to solid dosage forms that contain bupropion hydrochloride and glucono delta lactone or its corresponding open chain hydroxy acid derivative.

### BACKGROUND OF THE INVENTION

Bupropion hydrochloride is a well-known antidepressant and non-nicotine aid to smoking cessation. GlaxoSmithKline sells this drug product in the United States as WELLBUTRIN® (bupropion hydrochloride immediate release tablets), WELLBUTRIN® SR and ZYBAN® SR (bupropion hydrochloride sustained release tablets).

Bupropion hydrochloride itself is a water-soluble, crystalline solid that is highly hygroscopic and susceptible to decomposition. Because of the drug's instability, researchers working in this field have tried a number of different approaches to improve the storage stability of the drug in the formulation. Prior art patents variously describe the use of stabilizers to improve drug storage. For example, the disclosed stabilizers include: organic acids, carboxylic acids, dicarboxylic acids, inorganic acids, acid salts of amino acids, sodium metabisulfite, and sodium bisulfate. These prior art patents specifically describe the use of L-cysteine hydrochloride, glycine hydrochloride, malic acid, sodium metabisulphate, citric acid, tartaric acid, L-cystine dihydrochloride, oxalic acid, succinic acid, fumaric acid, phthalic acid, hydrochloric acid, phosphoric acid, nitric acid and sulphuric acid as stabilizers.

### SUMMARY OF THE INVENTION

In one general aspect there is provided a solid dosage form that includes bupropion hydrochloride; and a stabilizer. The stabilizer is glucono delta lactone or its corresponding open chain hydroxy acid derivative.

Embodiments of the solid dosage form may include one or more of the following features. For example, the bupropion hydrochloride may retain at least 80% of the bupropion hydrochloride potency after storage for three months at 40° C. and 75% relative humidity.

The stabilizer may be glucono delta lactone. The stabilizer may be a corresponding open chain hydroxy acid derivative of glucono delta lactone. The corresponding open chain hydroxy acid derivative of glucono delta lactone may be gluconic acid. The concentration of glucono delta lactone or corresponding open chain hydroxy derivative may be from about 5% to about 100% by weight of the bupropion hydrochloride, and may be about 5% to about 50% by weight of the bupropion hydrochloride.

The amount of bupropion hydrochloride may be between about 25 and about 500 mg w/w of the solid dosage form. The solid dosage form may be in the form of a tablet, a capsule, and a granulate with or without an immediate release profile, a modified release profile, or an extended release profile. The solid dosage form may be a tablet and the tablet may be a sustained release tablet. The solid dosage forms may be a capsule and the capsule may be a sustained release capsule.

The solid dosage form may further include one or more pharmaceutically acceptable excipients that include rate controlling polymers, diluents, binders, disintegrants, lubricants, glidants, and coloring agents. The release rate controlling polymers may be one or more of cellulose derivatives, acrylates, a mixture of polyvinylacetate and povidone, polyethylene oxides, starch and its derivatives, gums, alginates, carbohydrate based polymers, and polysaccharide. The cellulose derivative may be one or more of ethyl cellulose, methylcellulose, hydroxymethylcellulose, hydroxyethylcellulose, hydroxypropylcellulose, hydroxypropyl methylcellulose, and sodium carboxymethylcellulose and, in particular, may be hydroxypropyl cellulose.

The diluent may be microcrystalline cellulose and the lubricant may be stearic acid.

In another general aspect there is provided a process for preparing a solid dosage form of bupropion hydrochloride. The process includes mixing bupropion hydrochloride and a stabilizer to form a blend and orming the blend into a solid dosage form. The stabilizer may be glucono delta lactone or its corresponding open chain hydroxy acid derivative.

Embodiments of the process may include one or more of the following features. For example, the solid dosage form may retain at least 80% of the bupropion hydrochloride potency after storage for three months at 40° C. and 75% relative humidity.

The stabilizer may be glucono delta lactone. The stabilizer may be a corresponding open chain hydroxy acid derivative of glucono delta lactone. The corresponding open chain hydroxy acid derivative of glucono delta lactone may be gluconic acid. The concentration of glucono delta lactone or its corresponding open chain hydroxy derivative may be from between about 5% to about 100% by weight of bupropion hydrochloride. The concentration of glucono delta lactone or corresponding open chain hydroxy derivative may be from between about 5% to about 50% by weight of bupropion hydrochloride. The amount of bupropion hydrochloride may be from between about 25 to about 500 mg w/w of the solid dosage form.

In the process, shaping of the blend into a solid dosage form may include forming a tablet, capsule or granulate with or without an immediate release profile, a modified release profile, or an extended release profile. The solid dosage form may be a tablet and the tablet may have a sustained release profile. The solid dosage form may be a capsule and the capsule may have a sustained release profile.

The mixing may be one or more of wet granulation, dry granulation, and direct compression. The solid dosage form may further include one or more pharmaceutically acceptable excipients selected from rate controlling polymers, diluents, binders, disintegrants, lubricants, glidants and coloring agents. The release rate controlling polymers may include one or more of cellulose derivatives, acrylates, a mixture of polyvinlyacetate and povidone, polyethylene oxides, starch and their derivatives, gums, alginates, carbohydrate based polymers, and polysaccharide. The cellulose derivative may be one or more of ethyl cellulose, methylcellulose, hydroxymethylcellulose, hydroxyethylcellulose, hydroxypropylcellulose, hydroxypropyl methylcellulose, and sodium carboxymethylcellulose, and, in particular, the cellulose derivative may be hydroxypropyl cellulose.

The diluent may be microcrystalline cellulose and the lubricant may be stearic acid.

In another general aspect there is provided a method of treating either or both of depression and nicotine addiction in a human. The method includes orally administering to a human in need thereof a solid dosage form that includes bupropion hydrochloride and a stabilizer. The stabilizer is glucono delta lactone or its corresponding open chain hydroxy acid derivative.

Embodiments of the method may include any one or more of the following features or those described above. For example, the bupropion hydrochloride may retain at least 80% of the bupropion hydrochloride potency after storage for three months at 40° C. and 75% relative humidity. The stabilizer may be glucono delta lactone. The stabilizer may be a corresponding open chain hydroxy acid derivative of glucono delta lactone. The corresponding open chain hydroxy acid derivative of glucono delta lactone may be gluconic acid. The concentration of glucono delta lactone or corresponding open chain hydroxy derivative may be from about 5% to about 100% by weight of the bupropion hydrochloride and, in particular, may be from about 5% to about 50% by weight of the bupropion hydrochloride. The amount of bupropion hydrochloride may be between about 25 mg and about 500 mg w/w of the solid dosage form.

The solid dosage form may be one or more of a tablet, a capsule, and a granulate with or without an immediate release profile, a modified release profile, or an extended release profile.

The details of one or more embodiments of the inventions are set forth in the description below. Other features, objects and advantages of the inventions will be apparent from the description and claims.

### DETAILED DESCRIPTION OF THE INVENTION

We have now discovered that stable bupropion hydrochloride solid dosage forms can be prepared with glucono delta lactone or a corresponding open chain hydroxy acid derivative. Glucono delta lactone can be added to the dosage form as such or in the form of a corresponding open chain hydroxy acid derivative. Glucono delta lactone is a crystalline compound that hydrolyses to the corresponding open chain hydroxy acid derivative upon contact with moisture. The structure of glucono delta lactone is the following:

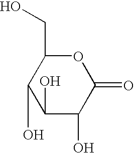

The term “bupropion hydrochloride” as used herein refers to the hydrochloride salt of m-chloro-α-(t-butylamino) propiophenone. The amount of bupropion hydrochloride may vary from between about 25 to about 500 mg w/w of the solid dosage form, although lower amounts are within the scope of the term when such amounts are therapeutically effective.

The glucono delta lactone described above can be added as such or as a corresponding open chain hydroxy acid derivative, i.e., gluconic acid. The addition of glucono delta lactone is preferred in some instances due to its ease of handling, sweet taste and high aqueous solubility. These stabilizers can be easily used in compositions prepared by, for example, either wet granulation or dry granulation methods.

These bupropion hydrochloride stabilizers can be used in a concentration, for example, which can effectively retain at least about 80% of the potency of bupropion hydrochloride in bupropion hydrochloride solid dosage forms after storage for three months at 40° C. and 75% relative humidity. Of course these concentrations can be varied either upward or downward depending upon the various standards, norms, and regulatory requirements of the country or agency reviewing or approving the drug. For example, the amount of glucono delta lactone or its corresponding open chain hydroxy acid derivative may vary from between about 5% to about 100% of the weight of the bupropion hydrochloride and, in particular, it may be between about 5% to 50% of the weight of bupropion hydrochloride.

The pharmaceutically acceptable excipients may be selected from one or more of rate controlling polymers (depending upon the choice of whether an instant or sustained release composition is being formulated), coating polymers, diluents, binders, disintegrants, lubricants, glidants and coloring agents compatible with bupropion hydrochloride.

The rate-controlling polymers may be a release rate controlling polymer and may be selected from one or more of any such pharmaceutically acceptable excipients that can control the rate of release of the active ingredient. In particular, such release rate-controlling polymers can be selected from one or more of cellulose derivatives, acrylates, methacrylates, polyvinlyacetate/povidone mixture, polyethylene oxides, starch and their derivatives, gums, alginates, carbohydrate based polymers, polysaccharides or combinations thereof.

The cellulose derivative can be selected from one or more of ethyl cellulose, methylcellulose, hydroxymethylcellulose, hydroxyethylcellulose, hydroxypropylcellulose, hydroxypropyl methylcellulose and sodium carboxymethylcellulose of different degree of substitution and molecular weights. These release rate-controlling polymers can be used alone or in combination. Various degrees of substitution and/or different molecular weights corresponding to a different degree of viscosity can be used as suitable cellulose based rate-controlling system.

The rate controlling polymer can be used in a concentration of between about 5% to about 60% w/w of the solid dosage form, depending on the polymer used. The use of hydroxypropyl methylcellulose (HPMC), hydroxypropylcellulose, a polyvinyl acetate/povidone mixture, or Carboxyvinyl polymers, such as Carbopol®, are preferred. Upon hydration, these polymers swell to form a gelatinous barrier through which either the drug may diffuse out, be released by erosion of the barrier, or a combination of erosion and diffusion.

Diluents may be selected from any pharmaceutically acceptable excipients that gives bulk to the composition and improves compressibility. For example, preferable diluents include one or more of starch or its derivatives, microcrystalline cellulose, lactose, glucose, mannitol, alginates, alkali earth metal salts, dicalcium phosphate, glyceryl monostearate, polyvinyl acetate/povidone mixture or polyethylene glycols.

Binders may be selected from any pharmaceutically acceptable excipients that have cohesive properties to act as a binder. For example, preferable excipients include one or more starch, gelatin, highly dispersed silica, mannitol, lactose, polyethylene glycol, polyvinylpyrrolidone, cross-linked polyvinylpyrrolidone, cross-linked carboxymethylcellulose, hydroxypropyl methylcellulose, hydroxypropyl cellulose and natural or synthetic gums.

The disintegrant may be selected from, for example, one or more of sodium starch glycolate, carboxy methylcellulose, croscarmellose sodium and crospovidone or combination thereof. Other suitable disintegrants also may be used separately or in combination.

Lubricants may be selected from, for example, one or more of talc, stearic acid, magnesium stearate, other alkali earth metal stearates such as calcium and zinc, sodium lauryl sulphate, hydrogenated vegetable oil, sodium benzoate, sodium stearyl fumarate, glyceryl monostearate and PEG 4000. Other suitable lubricants also may be used separately or in combination.

Glidants may be selected from, for example, colloidal silicon dioxide and talc, although any other suitable glidants may be used.

Solid dosage forms that include bupropion hydrochloride, stabilizer, and other excipients include tablets, caplets, capsules and granulates. These dosage forms may have immediate release, modified release and/or extended release profiles.

The stabilized dosage forms of bupropion hydrochloride can be conveniently prepared by any of the methods known to those skilled in the art. For tablets, the method of choice may be wet granulation, dry granulation or direct compression. These methods include the basic step of intimately mixing the stabilizer with bupropion hydrochloride along with other pharmaceutically acceptable excipients and shaping the product into a solid dosage form. Alternatively, the stabilizer (either the complete amount or a portion thereof) may also be added to the granulating fluid during wet granulation.

The stability of bupropion hydrochloride compositions was tested after storage for four to twelve weeks at 40° C. and 75% relative humidity. Bupropion hydrochloride compositions stored under these conditions retained at least 80% of the bupropion hydrochloride in the composition. In many instances, the formulations retained more than 85% of bupropion hydrochloride in the composition.

The present invention is further exemplified by, but is not intended to be limited to, the following examples:

### EXAMPLES 1 AND 2

### Bupropion Hydrochloride 150 mg Formulations (Low Glucono Delta Lactone Formulations)

Weight (mg) per tablet

| Ingredient | Example 1 | Example 2 |
|---|---|---|
| Bupropion hydrochloride | 150.00 | 150.00 |
| Hydroxypropyl cellulose | 50 | 50 |
| Microcrystalline cellulose | 208.5 | 168.5 |
| Glucono delta lactone | 3.5 | 3.5 |
| Polyvinlyacetate/Povidone | — | 40 |
| mixture | | |
| Stearic acid | 4 | 4 |
| Total | 416.00 | 416.00 |

The above bupropion hydrochloride formulations were prepared using the following process:
1. Bupropion hydrochloride, hydroxypropyl cellulose, microcrystalline cellulose, and the polyvinlyacetate/povidone mixture (in example 2) were mixed in a blender.
2. The blend of step 1 was granulated with an aqueous solution of glucono delta lactone to form granules.
3. The granules were dried and sized accordingly.
4. The dried and sized granules were lubricated with stearic acid and then compressed to form tablets.

### EXAMPLE 3

### Bupropion Hydrochloride 150 mg Formulation (High Glucono Delta Lactone Formulation)

| Ingredient | Weight (mg) per tablet |
|---|---|
| Bupropion hydrochloride | 150.00 |
| Hydroxypropyl cellulose | 50 |
| Microcrystalline cellulose | 168.5 |
| Glucono delta lactone | 43 |
| Stearic acid | 4 |
| Total | 416.00 |

Process:
1. Bupropion hydrochloride, hydroxypropyl cellulose, a first portion of the glucono delta lactone and the microcrystalline cellulose were mixed in a blender.
2. An aqueous solution of the remaining quantity of glucono delta lactone was used to granulate the blend of step 1.
3. The wet mass of step 2 was dried in a fluid bed dryer and the granules were sized.
4. The dried and sized granules were lubricated with stearic acid and then compressed into tablets.

Product stability data was obtained for the above formulation by storage at 40° C. and 75% relative humidity for three months. Potency was determined using HPLC. This product stability data is presented in Table 1.

TABLE 1
Comparative stability of bupropion hydrochloride tablets prepared as
per the composition of Examples 1-3 relative to commercially available
bupropion hydrochloride tablets (WELLBUTRIN SR ®).

| | % Bupropion hydrochloride* | | | |
|---|---|---|---|---|
| | EXAMPLES | | | |
| Stability conditions | 1 | 2 | 3 | WELLBUTRIN SR ® |
| Initial | 101.6 | 99.7 | 100.1 | 105.3 |
| 1 month at 40° C./75% RH | 93.8 | 91.6 | 96.7 | 95.1 |
| 2 month at 40° C./75% RH | 88.5 | 83.9 | 99.5 | 89.0 |
| 3 month at 40° C./75% RH | 81.3 | 76.1 | 89.9 | — |

RH = Relative Humidity
*% of added quantity

The above data indicates that glucono delta lactone effectively stabilizes bupropion hydrochloride tablets under various formulation conditions. In particular the data indicates the increased stability provided by increasing the amount of glucono delta lactone (Example 3).

While several particular forms of the inventions have been described, it will be apparent that various modifications and combinations of the inventions detailed in the text can be made without departing from the spirit and scope of the inventions. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

1. A solid dosage form comprising:
bupropion hydrochloride; and
a stabilizer, wherein the stabilizer comprises glucono delta lactone or its corresponding open chain hydroxy acid derivative.

2. The solid dosage form of claim 1, wherein the bupropion hydrochloride retains at least 80% of the bupropion hydrochloride potency after storage for three months at 40° C. and 75% relative humidity.

3. The solid dosage form of claim 1, wherein the stabilizer is glucono delta lactone.

4. The solid dosage form of claim 1, wherein the stabilizer is a corresponding open chain hydroxy acid derivative of glucono delta lactone.

5. The solid dosage form of claim 4, wherein the corresponding open chain hydroxy acid derivative of glucono delta lactone is gluconic acid.

6. The solid dosage form of claim 1, wherein the concentration of glucono delta lactone or corresponding open chain hydroxy derivative comprises from about 5% to about 100% by weight of the bupropion hydrochloride.

7. The solid dosage form of claim 1, wherein the concentration of glucono delta lactone or corresponding open chain hydroxy derivative comprises from about 5% to about 50% by weight of the bupropion hydrochloride.

8. The solid dosage form of claim 1, wherein the amount of bupropion hydrochloride comprises between about 25 and about 500 mg w/w of the solid dosage form.

9. The solid dosage form of claim 1, wherein the solid dosage form comprises one or more of a tablet, a capsule, and a granulate with or without an immediate release profile, a modified release profile, or an extended release profile.

10. (canceled)

11. (canceled)

12. (canceled)

13. (canceled)

14. (canceled)

15. (canceled)

16. (canceled)

17. (canceled)

18. (canceled)

19. (canceled)

20. A process for preparing a solid dosage form of bupropion hydrochloride, the process comprising;
mixing bupropion hydrochloride and a stabilizer to form a blend, wherein the stabilizer comprises glucono delta lactone or its corresponding open chain hydroxy acid derivative; and
forming the blend into a solid dosage form.

21. The process of claim 20, wherein the solid dosage form retains at least 80% of the bupropion hydrochloride potency after storage for three months at 40° C. and 75% relative humidity.

22. The process of claim 20, wherein the stabilizer is glucono delta lactone.

23. The process of claim 20, wherein the stabilizer is a corresponding open chain hydroxy acid derivative of glucono delta lactone.

24. The process of claim 23, wherein the corresponding open chain hydroxy acid derivative of glucono delta lactone is gluconic acid.

25. The process of claim 20, wherein the concentration of glucono delta lactone or corresponding open chain hydroxy derivative comprises from between about 5% to about 100% by weight of bupropion hydrochloride.

26. The process of claim 25, wherein the concentration of glucono delta lactone or corresponding open chain hydroxy derivative comprises from between about 5% to about 50% by weight of bupropion hydrochloride.

27. The process of claim 20, wherein the amount of bupropion hydrochloride comprises from between about 25 to about 500 mg w/w of the solid dosage form.

28. (canceled)

29. (canceled)

30. (canceled)

31. (canceled)

32. (canceled)

33. (canceled)

34. (canceled)

35. (canceled)

36. The process of claim 20, wherein the solid dosage form further comprises one or more pharmaceutically acceptable excipients selected from rate controlling polymers, diluents, binders, disintegrants, lubricants, glidants and coloring agents.

37. (canceled)

38. (canceled)

39. (canceled)

40. (canceled)

41. (canceled)

42. A method of treating either or both of depression and nicotine addiction in a human, the method comprising orally administering to a human in need thereof a solid dosage form comprising bupropion hydrochloride and a stabilizer, wherein the stabilizer comprises glucono delta lactone or its corresponding open chain hydroxy acid derivative.

43. (canceled)

44. The method of claim 42, wherein the stabilizer is glucono delta lactone.

45. The method of claim 42, wherein the stabilizer is a corresponding open chain hydroxy acid derivative of glucono delta lactone.

46. (canceled)

47. (canceled)

48. (canceled)

49. The method of claim 42, wherein the amount of bupropion hydrochloride comprises between about 25 and about 500 mg w/w of the solid dosage form.

50. (canceled)
